(19) United States
(12) Patent Application Publication
(10) Pub. No.: US 2004/0259125 A1
(43) Pub. Date: Dec. 23, 2004

(54) Methods, systems and apparatus for identifying genetic differences in disease and drug response

(75) Inventors: Umesh G. Bhatia, San Jose, CA (US); Nicholas Erndt, Los Altos Hills, CA (US); Wenjin Zheng, San Diego, CA (US); Julia Montgomery, Santa Cruz, CA (US); Marina Vainer, Fremont, CA (US); Victor Tong, Dublin, CA (US)

Correspondence Address: MANATT PHELPS AND PHILLIPS, ROBERT D. BECKER, 1001 PAGE MILL ROAD, BUILDING 2, PALO ALTO, CA, 94304, US

(73) Assignee: OMNI GENETICS, INC., MENLO PARK, CA

(21) Appl. No.: 10/787,967

(22) Filed: Feb. 25, 2004

Related U.S. Application Data
(60) Provisional application No. 60/450,606, filed on Feb. 26, 2003.

(51) Int. Cl. C12Q 1/68

(52) U.S. Cl. 435/6

(57) ABSTRACT

Methods for identifying genetic factors associated with, for example, drug response, disease, phenotype and/or behavior. Also provided are systems and apparatus for practicing the methods of the invention. The invention identifies new and informative polymorphisms (not yet identified and relevant to drug response, disease, behavior and/or phenotype) or mutations using a knockout/deletion/removal strategy. Knockout is followed by a step of capturing/trapping/enriching DNA fragments carrying informative polymorphisms or mutations. The advantages of the present invention are beneficial in, for instance, large-scale pharmacogenomics studies in patients undergoing testing in clinical trials or to find new genes associated with disease at relatively low cost.

This application claims the benefit of co-pending provisional application Ser. No. 60/450,606; filed Feb. 26, 2003, the entirety of which is incorporated herein by this reference.

### FIELD OF THE INVENTION

The present invention relates generally to the field of genetic analysis. More particularly, the invention described here provides methods, systems and apparatus for identifying genetic differences, such as, for example, differences in drug response or disease predisposition, in two or more individuals or in two or more populations.

### BACKGROUND OF THE INVENTION

As is well known in the art, the term Single Nucleotide Polymorphism (SNP) refers to genetic variation at the individual nucleotide level. Because the whole genome draft is now available, the application of mapping SNPs to genes and disease has become somewhat easier. Currently, several million putative SNPs have been identified by the Wellcome Trust, NIH and others. Approaches that take advantage of this vast amount of information to facilitate drug discovery are likely to become increasingly important in the future.

Current strategies for SNP discovery are largely divided into two main approaches: sequence specific and sequence nonspecific (11-14). The sequence nonspecific approach detects a SNP without knowing where it resides on the genome, using experimental methods such as electrophoresis or liquid chromatography. Sequence specific approaches are mainly used for genotyping.

There are three steps involved in known methods for sequence specific SNP genotyping. First, a large-scale effort is undertaken to sequence genomic DNA from different sources. By comparing sequences from different sources, large amounts of SNPs can be identified and archived. The second step typically involves a genetic study to identify candidate genes that associate with disease or other interesting traits and identify SNPs associated with these genes. Third, diagnostic methods can be developed using commercially available technologies for detecting the identified SNPs.

There are many SNP detection technologies available on the market. All the SNP detection technologies rely on already identified SNPs. For SNPs that have already been identified in the region of interest —several technologies can be used for its detection. Current analytical platforms include: 1) Separating fragments based on sizing and variation in conformation. 2) Hybridization and 3) Array and beads based assay. All of these platforms rely on knowing the sequence around the SNP.

Prior art SNP detection technologies suffer from a number of disadvantages. Among these, it has been acknowledged that current technologies for SNP detection are not sufficiently robust, scalable and sensitive. Methods for gene cloning that calculate allelic frequencies within groups of dissimilar phenotypes were previously developed using adapter linked amplification methods. In many cases, genetic traits may be determined by several different SNPs, each SNP only contributing very small amounts of effect to the traits. The association of the traits with genes takes many years of genetic study and it is hard to identify all the effectors for the traits. Without identifying all the SNPs for a particular trait, it is difficult to conduct genotyping. These limitations dramatically reduce their application in certain important areas.

The known methods described above require prior identification of the target SNP. This means that there has to be a huge amount of up-front genetic work needed to associate the SNP with a phenotype or genetic disorder. Once the SNP has been identified, then current technology can be used for its detection.

For genetic disorders that have no SNP associations, current approaches are not a viable solution. For example, if the Alzheimer's disease gene has not been identified, then it is not possible to use current SNP detection technology to test which genes are possibly altered in the putative Alzheimer's patient. Although high throughput technologies are currently available that can streamline the detection, they are still limited to certain amounts of SNPs that can be detected. Complex disorders or phenotypes are the product of several genes and sometimes hundreds of mutations. If all the loci information is not available or if the relevant SNPs are not known, then genotyping a small number of contributors proves to be futile.

In addition, current SNP detection technologies largely rely on high volume sequencing or array based scanning methods. These identified SNPs then are studied to find their association with phenotype of genetic disorders. Such a process may take a long time and can be very costly. Even with the currently identified several million SNPs in the human genome, it is hard to identify which single SNP contributes to a particular phenotype or genetic disorder. One study from the SNP Consortium indicates that one of the main reason that genotyping has not been widely used is its high cost.

It can thus be seen that the art is in need of SNP detection technologies that provide cost-effective, high throughput SNP screening capacity without the need for prior SNP identification.

### BRIEF SUMMARY OF THE INVENTION

In accordance with a preferred embodiment, the present invention provides methods, systems and apparatus for identifying genetic factors that are associated with, for example, disease, differential drug response, behavior and phenotype. The method reads these factors directly from DNA fragments, genomic DNA, cDNA, cloned genes, or other genetic material from individuals or populations. The method makes no use of and in fact obviates existing array technologies.

The first step in the method according to the preferred embodiment is to take an individual's DNA and knockout/remove/destroy/delete all mismatches and polymorphisms, e.g., FIG. 1. We term this method knockout (FIG. 1). The strategy is based on the notion that there are millions of variations that are not relevant in one individual or group or population. For example, if a drug response is being investigated and if two groups are being examined: (A) responds well to a drug x; (B) has a serious side effect. Then both A and B will likely share millions of variations that have nothing to do with group B's adverse response to drug x. Given homozygous SNPs or mutations, the knockout process reduces the background noise and creates close to a “bare genome” containing little or no SNPs (FIG. 2-step 2). As used herein, the term “knockout DNA” means DNA that is substantially devoid of polymorphisms.

Knockout DNA is created by first digesting the DNA with four cutter restriction enzymes to generate fragments with an average size of 300 to 400 bp. DNA is then subjected to denaturation ->reannealing cycles followed by treatment with endonucleases to specifically remove the mismatched nucleotides regions in DNA heteroduplexes (1-3). In this process, the DNA surrounding the mismatches are not cleaved. If a group or population with a particular phenotype is under study, pooling their DNA should be avoided because alleles that occur at high frequencies tend to reduce or eliminate the alleles at low frequencies as the number of knockout cycles progresses.

The next step in the method involves capture (FIG. 1), selection or enrichment of double stranded DNA (dsDNA). The basic premise of this step is that polymorphisms or mutations relevant to the disease or drug response are likely to be present at different frequencies in individuals with a particular phenotype under investigation. For example, a group of individuals that respond adversely to drug x are likely going to have some alleles that predispose to the phenotype at higher/lower frequencies than individuals that respond well or show no response to drug x. We expect to see an inverse in the allelic frequencies in two populations that show a dramatically different phenotype.

### BRIEF DESCRIPTION OF THE DRAWINGS

The nature of preferred embodiment of the present invention as well as other embodiments of the present invention may be more clearly understood by reference to the following detailed description of the invention, to the appended claims, and to the drawings herein, in which:

FIG. 1 is a graphic representation of a knockout and capture method;

FIG. 2. is a flowchart illustrating the steps for identifying homozygous inherited polymorphisms and mutations; and

FIG. 3. is a flowchart illustrating the steps for identifying heterozygous inherited polymorphisms and mutations.

### DETAILED DESCRIPTION OF PREFERRED EMBODIMENT OF THE INVENTION

The present invention provides methods, systems and apparatus for identifying genetic factors that are associated with, for example, disease, differential drug response, behavior and phenotype. A method according to the invention reads these factors directly from DNA fragments, genomic DNA, CDNA, cloned genes, or other genetic material from individuals or populations.

Because there are over 7.5 million SNPs in an individual, the task of finding the relevant polymorphisms (e.g. 5-50) in disease or drug response has been a very daunting task. The method herein reduces the number of SNPs from millions to hundred or less candidates as illustrated below:

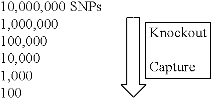

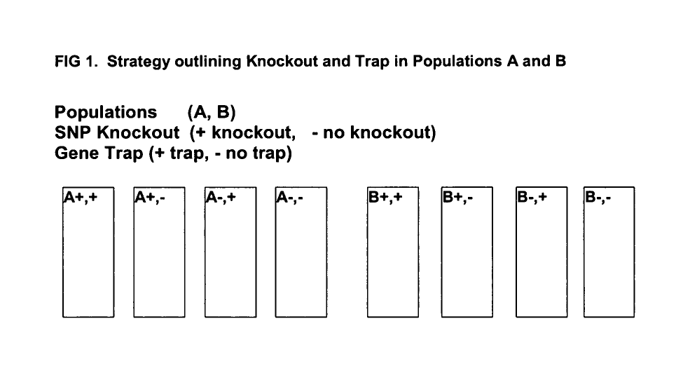

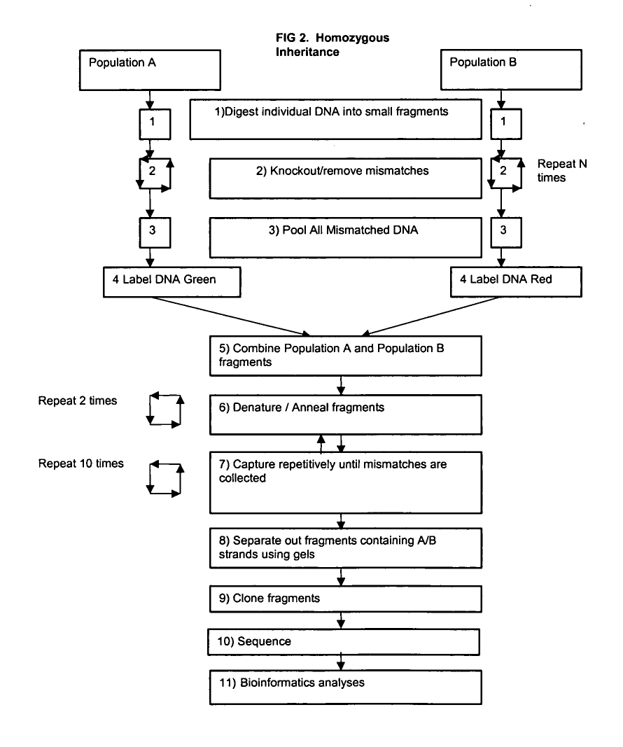

The first novel step in the process is to take an individual's DNA and knockout/remove/destroy/delete all mismatches and polymorphisms. We term this method knockout (FIG. 1). The strategy is based on the notion that there are millions of variations that are not relevant in one individual or group or population. For example, if a drug response is being investigated and if two groups are being examined: (A) responds well to a drug x; (B) has a serious side effect. Then both A and B will likely share millions of variations that have nothing to do with group B's adverse response to drug x. Given homozygous SNPs or mutations, the knockout process reduces the background noise and creates close to a “bare genome” containing little or no SNPs (FIG. 2-step 2) (i.e., “knockout DNA”).

Knockout DNA is created by first digesting the DNA with four cutter restriction enzymes to generate fragments with an average size of 300 to 400 bp. DNA is then subjected to denaturation ->reannealing cycles followed by treatment with endonucleases to specifically remove the mismatched nucleotides regions in DNA heteroduplexes (1-3). In this process, the DNA surrounding the mismatches are not cleaved. If we are looking at a group or population with a particular phenotype, we avoid pooling their DNA because alleles that occur at high frequencies tend to reduce or eliminate the alleles at low frequencies as the number of knockout cycles progresses.

The next step in the method involves capture (FIG. 1), selection or enrichment of double stranded DNA (dsDNA). The basic premise of this step is that polymorphisms or mutations relevant to the disease or drug response are likely to be present at different frequencies in individuals with a particular phenotype under investigation. For example, a group of individuals that respond adversely to drug x are likely going to have some alleles that predispose to the phenotype at higher/lower frequencies than individuals that respond well or show no response to drug x. We expect to see an inverse in the allelic frequencies in two populations that show a dramatically different phenotype.

The capture method is based on rules. For example:

If gene X in population A and gene X in population B have no variations =>no capture

If gene X in population A has variation (i) and if (i) is not in gene X in population B =>capture

If gene X in population A does not have variation (j) and if (j) is in gene X in population B=>capture

For homozygous traits (FIG. 2-step 7), the gene capture step is carried out post knockout by pooling DNA from two individuals, groups or populations. The pooled DNA is then subjected to cycles of denaturation and reannealing. Between each cycle, the dsDNA that harbor mismatches are captured. The capture step can be accomplished, for example, using DNA repair proteins (e.g. muts) that bind to DNA fragments with mismatches are used for the gene capture step (4,5). Muts is a product commercially available through Genecheck Inc. (Boulder, Colo.). The procedure involves spotting the protein on a membrane or plate or microbead followed by introduction of dsDNA fragments to the protein bound surface. Bound mismatched DNA is then eluted for further characterization (e.g. sequencing).

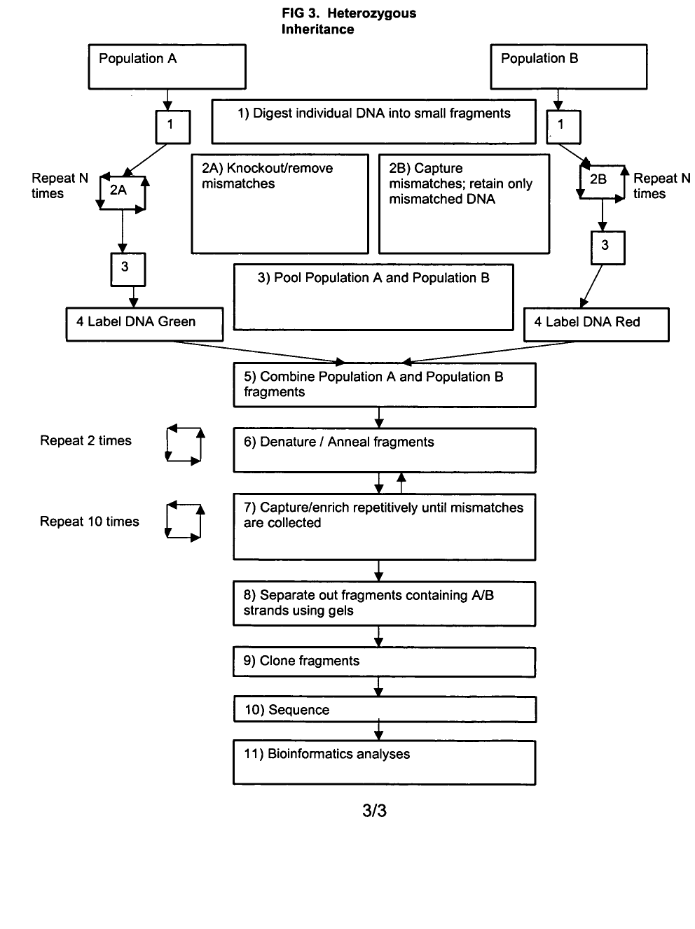

Heterozygous inherited polymorphisms or mutations are identified by repeat capture cycles (FIG. 3-step 2 and 7). For example, if groups A and B (good responders or adverse responders to drug x, respectively) are being investigated then each group is individually subjected to repeat capture cycles. Captured DNA fragments from group B are then combined with eluted DNA from group A and subject to further rounds of capture. The final captured DNA is then further analyzed.

The captured heteroduplex fragments are then separated based on molecular weight and sequence using 2 Dimensional Gradient Gel Electrophoresis (2DGGE) (6-10). Population A to Population B heteroduplexes are distinguished from heteroduplexes derived only from a single population using the dual labeling strategy outlined in FIGS. 2 and 3. Heteroduplexes from a single population will display a single color, heteroduplexes from both populations will display both colors. The Population A to Population B double stranded DNA fragments are then eluted from the polyacrylamide gradient gel using standard technologies and placed in separate wells of a microtiter plate.

The DNA ends are then polished and cloned into an appropriate vector which is also linearized with blunt ends. The cloned DNAs are then transformed into ‘E. coli’ and grown up and sequenced using standard technologies.

The DNA sequences are then compared with the public domain information using BLAST and relevant information concerning the fragments are acquired (i.e. identify gene families, locations, disease associations, etc.). This can verify existing known gene associations and determine unique gene associations.

Reference is now made to FIG. 1, which depicts a preferrred knockout and capture method according to the present invention. Two individuals or populations A and B are depicted [A, B]. A and B are either subject to knockout [A+ and B+] or no knockout [A− and B−]. A and B then undergo DNA capture [A++, B++, A−+, and B−+] or no capture [A+−, B+−, A−−, B−−].

FIG. 2. provides a flowchart illustrating steps for identifying homozygous inherited polymorphisms and mutations in populations A and B.

For Population A (e.g., positive responders):

Collect individual DNA for each person in population (step 1);

Digest DNA into small fragments using a four base cutter e.g. SAU3A (step 1);

Knockout/delete/remove mismatches in the fragments (step 2);

Pool DNA from Population A (step 3);

End label fragments to as to distinguish the two populations (i.e. Red fluor for Population A and green fluor for Population B) (step 4);

For Population B (e.g., negative responders)

Collect individual DNA for each person in population (step 1);

Digest DNA into small fragments using a four base cutter e.g. SAU3A (step 1);

Knockout/delete/remove mismatches in the fragments (step 2);

Pool DNA from Population B (step 3);

End label fragments to as to distinguish the two populations (i.e. Red fluor for Population A and green fluor for Population B) (step 4);

Combine Population A and Population B fragments (step 5);

Denature/Anneal fragments (step 6);

Capture repetitively until mismatches are collected (step 7);

Separate out fragments containing A/B strands using gels (step 8);

Clone fragments (step 9);

Sequence fragments (step 10); and

Bioinformatics analyses (step 11).

FIG. 3 illustrates the steps to identify heterozygous inherited polymorphisms and mutations in populations A and B.

For Population A (positive responders):

Collect individual DNA for each person in population (step 1)

Digest DNA into fragments using a four base cutter e.g. SAU3A (step 1)

Capture mismatch DNA for each individual repetitively until all mismatches are trapped; retain only those fragments that have no mismatches or Knockout/delete/remove mismatches in the fragments (step 2);

Pool DNA from group that have the same phenotype (step 3)

Label all fragments to as to distinguish the two populations (i.e. Red fluor for Population A and green fluor for Population B) (step 4)

For population B (negative responders):

Collect individual DNA for each person in population (step 1)

Digest DNA into fragments using a four base cutter e.g. SAU3A (step 1)

Capture mismatched DNA for each individual repetitively until all mismatches are collected fragments are collected (step 2)

Pool mismatched DNA from Population B that has the same phenotype (step 3)

Label all fragments to as to distinguish the two populations (i.e. Red fluor for Population A and green fluor for Population B) (step 4)

Combine Population A and Population B fragments (step 5)

Denature/Anneal fragments (step 6)

Capture mismatches repetitively until mismatches are collected (step 7)

Separate out fragments containing A/B strands using DNA gels (step 8)

Clone fragments (step 9)

Sequence fragments (step 10)

Bioinformatics analyses (step 11)

Although the present invention has been shown and described with reference to particular preferred embodiments, various additions, deletions and modifications that are obvious to a person skilled in the art to which the invention pertains, even if not shown or specifically described herein, are deemed to lie within the scope of the invention as encompassed by the following claims.

We claim:

1. A method for identifying at least one homozygous genetic factor associated with a first phenotype that differs from a second phenotype, wherein an association between the at least one homozygous genetic factor and the first phenotype is unknown, comprising:
providing at least one strand of first-phenotype knockout DNA substantially devoid of polymorphisms and at least one strand of second-phenotype knockout DNA substantially devoid of polymorphisms;
performing a denaturing/reannealing cycle on a pool comprising the at least one strand of first-phenotype knockout DNA and the at least one strand of second-phenotype knockout DNA; and
capturing at least one mismatch in the pool following the denaturing/reannealing cycle, the captured at least one mismatch representing the at least one homozygous genetic factor associated with the first phenotype.

2. The method according to claim 1, wherein providing at least one strand of first-phenotype knockout DNA comprises:
providing at least one strand of first-phenotype DNA;
digesting the at least one strand of first-phenotype DNA with four cutter restriction enzymes to produce a plurality of first-phenotype DNA fragments;
performing at least one cycle of denaturing/reannealing on the plurality of first-phenotype DNA fragments;
treating a plurality of reannealed first-phenotype DNA fragments with at least one endonuclease such that one or more mismatched DNA regions in the plurality of reannealed first-phenotype DNA fragments are removed without modifying DNA sequences adjacent the one or more mismatched DNA regions to produce the at least one strand of first-phenotype knockout DNA substantially devoid of polymorphisms.

3. The method according to claim 1, wherein providing the at least one strand of second-phenotype knockout DNA comprises:
providing at least one strand of second-phenotype DNA;
digesting the at least one strand of second-phenotype DNA with four cutter restriction enzymes to produce a plurality of second-phenotype DNA fragments;
performing at least one cycle of denaturing/reannealing on the plurality of second-phenotype DNA fragments;
treating a plurality of reannealed second-phenotype DNA fragments with at least one endonuclease such that one or more mismatched DNA regions in the plurality of reannealed second-phenotype DNA fragments are removed without modifying DNA sequences adjacent the one or more mismatched DNA regions to produce the at least one strand of second-phenotype knockout DNA substantially devoid of polymorphisms.

4. The method according to claim 1, further comprising:
performing a subsequent denaturing/reannealing cycle on a subsequent pool comprising the at least one strand of uncaptured first-phenotype knockout DNA and the at least one strand of uncaptured second-phenotype knockout DNA; and
capturing at least one mismatch in the subsequent pool following the subsequent denaturing/reannealing cycle, the at least one mismatch representing the at least one homozygous genetic factor associated with the first phenotype.

5. The method according to claim 2, wherein the plurality of first-phenotype DNA fragments average less than approximately 500 base pairs long.

6. The method according to claim 5, wherein the plurality of first-phenotype DNA fragments average approximately 300-400 base pairs long.

7. The method according to claim 3, wherein the plurality of second-phenotype DNA fragments average less than approximately 500 base pairs long.

8. The method according to claim 7, wherein the plurality of second-phenotype DNA fragments average approximately 300-400 base pairs long.

9. The method according to claim 1, wherein the first phenotype is at least one of a disease predisposition, a disease susceptibility, a drug reaction predisposition and a behavior predisposition.

10. The method according to claim 1, wherein the at least one homozygous genetic factor comprises a polymorphism.

11. The method according to claim 1, wherein the at least one homozygous genetic factor comprises a single nucleotide polymorphism.

12. The method according to claim 1, wherein the at least one homozygous genetic factor comprises a plurality of polymorphisms.

13. The method according to claim 1, wherein the at least one homozygous genetic factor comprises a plurality of single nucleotide polymorphisms.

14. The method according to claim 1, wherein capturing at least one mismatch comprises exposing at least one mismatch to a DNA repair protein.

15. The method according to claim 14, wherein the DNA repair protein is of a type that binds to mismatched DNA.

16. The method according to claim 14, wherein the DNA repair protein is a MutS protein.

17. The method according to claim 1, further comprising differentially labeling the at least one strand of first-phenotype knockout DNA and the at least one strand of second-phenotype knockout DNA.

18. The method according to claim 1, further comprising characterizing the captured at least one mismatch.

19. The method according to claim 18, wherein characterizing the captured at least one mismatch comprises sequencing the at least one mismatch.

20. The method according to claim 19, further comprising performing bioinformatic analysis on the mismatch.

21. A method for diagnosing a disease associated with at least one homozygous genetic factor, comprising identifying the at least one homozygous genetic factor associated with the disease by the method according to claim 1.

22. A method for predicting a drug response associated with at least one homozygous genetic factor, comprising identifying the at least one homozygous genetic factor associated with the drug response by the method according to claim 1.

23. A method for predicting a disease predisposition associated with at least one homozygous genetic factor, comprising identifying the at least one homozygous genetic factor associated with the disease predisposition by the method according to claim 1.

24. A method for predicting a disease susceptibility associated with at least one homozygous genetic factor, comprising identifying the at least one homozygous genetic factor associated with the disease susceptibility by the method according to claim 1.

25. A method for identifying at least one heterozygous genetic factor associated with a first phenotype that differs from a second phenotype, wherein an association between the at least one heterozygous genetic factor and the first phenotype is unknown, comprising:
providing at least one strand of first-phenotype capture DNA enriched for polymorphisms of interest and at least one strand of second-phenotype knockout DNA substantially devoid of polymorphisms;
performing a denaturing/reannealing cycle on a pool comprising at least one strand of first-phenotype capture DNA and at least one strand of second-phenotype knockout DNA; and
capturing at least one mismatch in the pool following the denaturing/reannealing cycle, the at least one mismatch representing the at least one heterozygous genetic factor associated with the first phenotype.

26. The method according to claim 25, wherein providing at least one strand of first-phenotype capture DNA comprises:
providing at least one strand of first-phenotype DNA;
digesting the at least one strand of first-phenotype DNA with four cutter restriction enzymes to produce a plurality of first-phenotype DNA fragments; and
capturing a plurality of first-phenotype mismatched DNA fragments in at least one cycle such that one or more mismatched DNA regions in the plurality of first-phenotype DNA fragments is retained to produce at least one strand of first-phenotype capture DNA enriched with polymorphisms of interest.

27. The method according to claim 25, wherein providing at least one strand of second-phenotype knockout DNA comprises:
providing at least one strand of second-phenotype DNA;
digesting at least one strand of second-phenotype DNA with four cutter restriction enzymes to produce a plurality of second-phenotype DNA fragments;
performing at least one cycle of denaturing/reannealing on the plurality of second-phenotype DNA fragments;
treating a plurality of reannealed second-phenotype DNA fragments with at least one endonuclease such that one or more mismatched DNA regions in the plurality of reannealed second-phenotype DNA fragments are removed without modifying DNA sequences adjacent the one or more mismatched DNA regions to produce at least one strand of second-phenotype knockout DNA substantially devoid of polymorphisms.

28. The method according to claim 25, further comprising:
performing a subsequent denaturing/reannealing cycle on a subsequent pool comprising at least one strand of uncaptured first-phenotype capture DNA and at least one strand of uncaptured second-phenotype knockout DNA; and
capturing at least one mismatch in the subsequent pool following the subsequent denaturing/reannealing cycle, at least one mismatch representing at least one heterzygous genetic factor associated with the first phenotype.

29. The method according to claim 27, wherein the plurality of second-phenotype DNA fragments average less than approximately 500 base pairs long.

30. The method according to claim 29, wherein the plurality of second-phenotype DNA fragments average approximately 300-400 base pairs long.

31. The method according to claim 25, wherein the first phenotype comprises at least one of a disease predisposition, a disease susceptibility, a drug reaction predisposition and a behavior predisposition.

32. The method according to claim 25, wherein at least one heterozygous genetic factor comprises a polymorphism.

33. The method according to claim 25, wherein at least one heterozygous genetic factor comprises a single nucleotide polymorphism.

34. The method according to claim 25, wherein at least one heterozygous genetic factor comprises a plurality of polymorphisms.

35. The method according to claim 25, wherein at least one heterozygous genetic factor comprises a plurality of single nucleotide polymorphisms.

36. The method according to claim 25, wherein capturing at least one mismatch comprises exposing the at least one mismatch to a DNA repair protein.

37. The method according to claim 36, wherein the DNA repair protein is of a type that binds to mismatched DNA.

38. The method according to claim 37, wherein the DNA repair protein is a MutS protein.

39. The method according to claim 25, further comprising differentially labeling the at least one strand of first-phenotype capture DNA and the at least one strand of second-phenotype knockout DNA.

40. The method according to claim 25, further comprising characterizing the captured at least one mismatch.

41. The method according to claim 40, wherein characterizing the captured at least one mismatch comprises sequencing the at least one mismatch.

42. The method according to claim 41, further comprising performing bioinformatic analysis on the captured at least one mismatch.

43. A method for diagnosing a disease associated with at least one heterozygous genetic factor, comprising identifying the at least one heterozygous genetic factor associated with the disease by the method according to claim 25.

44. A method for predicting a drug response associated with at least one heterozygous genetic factor, comprising identifying the at least one heterozygous genetic factor associated with the drug response by the method according to claim 25.

45. A method for predicting a disease predisposition associated with at least one heterozygous genetic factor, comprising identifying the at least one heterozygous genetic factor associated with the disease predisposition by the method according to claim 25.

46. A method for predicting a disease susceptibility associated with at least one heterozygous genetic factor, comprising identifying the at least one heterozygous genetic factor associated with the disease susceptibility by the method according to claim 25.

47. An apparatus for identifying at least one homozygous genetic factor associated with a first phenotype that differs from a second phenotype, wherein an association between the at least one homozygous genetic factor and the first phenotype is unknown, the apparatus comprising:
a knockout DNA-producing component for producing at least one strand of first-phenotype knockout DNA substantially devoid of polymorphisms and at least one strand of second-phenotype knockout DNA substantially devoid of polymorphisms;
a DNA denaturing/reannealing component for performing a denaturing/reannealing cycle on a pool comprising at least one strand of first-phenotype knockout DNA and at least one strand of second-phenotype knockout DNA; and
a capture component for capturing at least one mismatch in the pool following the denaturing/reannealing cycle, the captured at least one mismatch representing at least one homozygous genetic factor associated with the first phenotype.

48. A system for identifying at least one homozygous genetic factor associated with a first phenotype that differs from a second phenotype, wherein an association between the at least one homozygous genetic factor and the first phenotype is unknown, the system comprising:
a knockout DNA-producing component for producing at least one strand of first-phenotype knockout DNA substantially devoid of polymorphisms and at least one strand of second-phenotype knockout DNA substantially devoid of polymorphisms;
a DNA denaturing/reannealing component for performing a denaturing/reannealing cycle on a pool comprising at least one strand of first-phenotype knockout DNA and at least one strand of second-phenotype knockout DNA;
a capture component for capturing at least one mismatch in the pool following the denaturing/reannealing cycle, the captured at least one mismatch representing at least one homozygous genetic factor associated with the first phenotype; and
a processing component in communication with the knockout DNA-producing component, the DNA denaturing/reannealing component, and the capture component, the processing component providing monitoring and control of the functions of the knockout DNA-producing component, the DNA denaturing/reannealing component, and the capture component.

49. An apparatus for identifying at least one heterozygous genetic factor associated with a first phenotype that differs from a second phenotype, wherein an association between the at least one heterozygous genetic factor and the first phenotype is unknown, the apparatus comprising:
a capture DNA/knockout DNA-producing component for producing at least one strand of first-phenotype capture DNA enriched for polymorphisms of interest and at least one strand of second-phenotype knockout DNA substantially devoid of polymorphisms;
a DNA denaturing/reannealing component for performing a denaturing/reannealing cycle on a pool comprising at least one strand of first-phenotype capture DNA and at least one strand of second-phenotype knockout DNA; and
a capture component for capturing at least one mismatch in the pool following the denaturing/reannealing cycle, the captured at least one mismatch representing at least one heterozygous genetic factor associated with the first phenotype.

50. A system for identifying at least one heterozygous genetic factor associated with a first phenotype that differs from a second phenotype, wherein an association between the at least one heterozygous genetic factor and the first phenotype is unknown, the system comprising:
a capture DNA/knockout DNA-producing component for producing at least one strand of first-phenotype capture DNA enriched for polymorphisms of interest and at least one strand of second-phenotype knockout DNA substantially devoid of polymorphisms;
a DNA denaturing/reannealing component for performing a denaturing/reannealing cycle on a pool comprising at least one strand of first-phenotype capture DNA and at least one strand of second-phenotype knockout DNA;
a capture component for capturing at least one mismatch in the pool following the denaturing/reannealing cycle, the captured at least one mismatch representing at least one heterozygous genetic factor associated with the first phenotype; and
a processing component in communication with the knockout DNA-producing component, the DNA denaturing/reannealing component, and the capture component, the processing component providing monitoring and control of the functions of the knockout DNA-producing component, the DNA denaturing/reannealing component, and the capture component.

51. The method according to claim 1, wherein providing at least one strand of first-phenotype knockout DNA substantially devoid of polymorphisms and the at least one strand of second-phenotype DNA substantially devoid of polymorphisms comprises providing DNA fragments, genomic DNA, cDNA, cloned genes, or other genetic material.

52. The method according to claim 25, wherein providing at least one strand of first-phenotype capture DNA enriched for polymorphisms of interest and at least one strand of second-phenotype knockout DNA substantially devoid of polymorphisms comprises providing DNA fragments, genomic DNA, cDNA, cloned genes, or other genetic material.
